SEQUENCE LISTING (21 sequences)

SEQ ID NO: 1 - DNA, 2589 bp, Homo sapiens
  misc_feature (2498)..(2498): n is a, c, g, or t
aagcagtggtaacaacgcagagtacgcgggggtataaagcgagcgcgctgaccccggcatgtccaggatgttaccgttcctgctgctgctctggtttctgcccatcactgaggggtcccagcgggctgaacccatgttcactgcagtcaccaactcagttctgcctcctgactatgacagtaatcccacccagctcaactatggtgtggcagttactgatgtggaccatgatggggactttgagatcgtcgtggcggggtacaatggacccaacctggttctgaagtatgaccgggcccagaagcggctggtgaacatcgcggtcgatgagcgcagctcaccctactacgcgctgcgggaccggcaggggaacgccatcggggtcacagcctgcgacatcgacggggacggccgggaggagatctacttcctcaacaccaataatgccttctcgggggtggccacgtacaccgacaagttgttcaagttccgcaataaccggtgggaagacatcctgagcgatgaggtcaacgtggcccgtggtgtggccagcctctttgccggacgctctgtggcctgtgtggacagaaagggctctggacgctactctatctacattgccaattacgcctacggtaatgtgggccctgatgccctcattgaaatggaccctgaggccagtgacctctcccggggcattctggcgctcagagatgtggctgctgaggctggggtcagcaaatatacagggggccgaggcgtcagcgtgggccccatcctcagcagcagtgcctcggatatcttctgcgacaatgagaatgggcctaacttccttttccacaaccggggcgatggcacctttgtggacgctgcggccagtgctggtgtggacgacccccaccagcatgggcgaggtgtcgccctggctgacttcaaccgtgatggcaaagtggacatcgtctatggcaactggaatggcccccaccgcctctatctgcaaatgagcacccatgggaaggtccgcttccgggacatcgcctcacccaagttctccatgccctcccctgtccgcacggtcatcaccgccgactttgacaatgaccaggagctggagatcttcttcaacaacattgcctaccgcagctcctcagccaaccgcctcttccgcgtcatccgtagagagcacggagaccccctcatcgaggagctcaatcccggcgacgccttggagcctgagggccggggcacagggggtgtggtgaccgacttcgacggagacgggatgctggacctcatcttgtcccatggagagtccatggctcagccgctgtccgtcttccggggcaatcagggcttcaacaacaactggctgcgagtggtgccacgcacccggtttggggcctttgccaggggagctaaggtcgtgctctacaccaagaagagtggggcccacctgaggatcatcgacgggggctcaggctacctgtgtgagatggagcccgtggcacactttggcctggggaaggatgaagccagcagtgtggaggtgacgtggccagatggcaagatggtgagccggaacgtggccagcggggagatgaactcagtgctggagatcctctacccccgggatgaggacacacttcaggacccagccccactggagtgtggccaaggattctcccagcaggaaaatggccattgcatggacaccaatgaatgcatccagttcccattcgtgtgccctcgagacaagcccgtatgtgtcaacacctatggaagctacaggtgccggaccaacaagaagtgcagtcggggctacgagcccaacgaggatggcacagcctgcgtggggactctcggccagtcaccgggcccccgccccaccacccccaccgctgctgctgccactgccgctgctgctgccgctgctggagctgccactgctgcaccggtcctcgtagatggagatctcaatctggggtcggtggttaaggagagctgcgagcccagctgctgagcaggggtgggacatgaaccagcggatggagtccagcaggggagtgggaaagtgggcttgtgctgctgcctagacagtagggatgtaaaggcctgggagctagaccctccccaagcccatccatgcacattacttagctaacaattagggagactcgtaaggccaggccctgtgctgggcacatagctgtgatcacagcagacagggtcgctgccctgatggcgcttacattccagtgggtctaatgaccatatcttaggacacagatgtgcccagggaggtggtgtcactgcacaggaagtatgaggactttagtgtcctgagttcaaatcctgattcaggaactcacaaagctatgtgaccttacaccagtcacttaacttgttagccatccattatcgcatctgcaaaatggggattaagaatagaatcttggggttagtgtggagattagattaaatgtatgtaagacacttancacaaaacctggcacatagtaaaggctcaataaaaacaagtgcctctcactgggctttgtcaacacgtcaaaaaaaaaaaaaaaaaaaaaa

SEQ ID NO: 2 - protein, 653 aa, Homo sapiens
MSRMLPFLLLLWFLPITEGSQRAEPMFTAVTNSVLPPDYDSNPTQLNYGVAVTDVDHDGDFEIVVAGYNGPNLVLKYDRAQKRLVNIAVDERSSPYYALRDRQGNAIGVTACDIDGDGREEIYFLNTNNAFSGVATYTDKLFKFRNNRWEDILSDEVNVARGVASLFAGRSVACVDRKGSGRYSIYIANYAYGNVGPDALIEMDPEASDLSRGILALRDVAAEAGVSKYTGGRGVSVGPILSSSASDIFCDNENGPNFLFHNRGDGTFVDAAASAGVDDPHQHGRGVALADFNRDGKVDIVYGNWNGPHRLYLQMSTHGKVRFRDIASPKFSMPSPVRTVITADFDNDQELEIFFNNIAYRSSSANRLFRVIRREHGDPLIEELNPGDALEPEGRGTGGVVTDFDGDGMLDLILSHGESMAQPLSVFRGNQGFNNNWLRVVPRTRFGAFARGAKVVLYTKKSGAHLRIIDGGSGYLCEMEPVAHFGLGKDEASSVEVTWPDGKMVSRNVASGEMNSVLEILYPRDEDTLQDPAPLECGQGFSQQENGHCMDTNECIQFPFVCPRDKPVCVNTYGSYRCRTNKKCSRGYEPNEDGTACVGTLGQSPGPRPTTPTAAAATAAAAAAAGAATAAPVLVDGDLNLGSVVKESCEPSC

SEQ ID NO: 3 - DNA, 20 bp, Homo sapiens
ttgccaattacgcctacggt

SEQ ID NO: 4 - DNA, 21 bp, Homo sapiens
cctggtcattgtcaaagtcgg

SEQ ID NO: 5 - DNA, 25 bp, Homo sapiens
cccttcattgacctcaactacatgg

SEQ ID NO: 6 - DNA, 25 bp, Homo sapiens
ggtccaccaccctgttgctgtagcc

SEQ ID NO: 7 - DNA, 25 bp, Homo sapiens
gggtccattgtaccccgccacgacg

SEQ ID NO: 8 - DNA, 22 bp, Homo sapiens
ctcaaagtccccatcatggtcc

SEQ ID NO: 9 - DNA, 23 bp, Homo sapiens
ctcagccgctgtccgtcttccgg

SEQ ID NO: 10 - DNA, 22 bp, Homo sapiens
gcttcaacaacaactggctgcg

SEQ ID NO: 11 - DNA, 26 bp, Homo sapiens
ggatgggcttggggagggtctagctc

SEQ ID NO: 12 - DNA, 22 bp, Homo sapiens
gcagcagcacaagcccactttc

SEQ ID NO: 13 - DNA, 24 bp, Homo sapiens
gtgcccagggaggtggtgtcactg

SEQ ID NO: 14 - DNA, 26 bp, Homo sapiens
gcacaggaagtatgaggactttagtg

SEQ ID NO: 15 - DNA, 27 bp, Homo sapiens
gggatttaaagtgcatataactgaagg

SEQ ID NO: 16 - DNA, 2039 bp, Homo sapiens
aagcagtggtaacaacgcagagtacgcgggggtataaagcgagcgcgctgaccccggcatgtccaggatgttaccgttcctgctgctgctctggtttctgcccatcactgaggggtcccagcgggctgaacccatgttcactgcagtcaccaactcagttctgcctcctgactatgacagtaatcccacccagctcaactatggtgtggcagttactgatgtggaccatgatggggactttgagatcgtcgtggcggggtacaatggacccaacctggttctgaagtatgaccgggcccagaagcggctggtgaacatcgcggtcgatgagcgcagctcaccctactacgcgctgcgggaccggcaggggaacgccatcggggtcacagcctgcgacatcgacggggacggccgggaggagatctacttcctcaacaccaataatgccttctcgggggtggccacgtacaccgacaagttgttcaagttccgcaataaccggtgggaagacatcctgagcgatgaggtcaacgtggcccgtggtgtggccagcctctttgccggacgctctgtggcctgtgtggacagaaagggctctggacgctactctatctacattgccaattacgcctacggtaatgtgggccctgatgccctcattgaaatggaccctgaggccagtgacctctcccggggcattctggcgctcagagatgtggctgctgaggctggggtcagcaaatatacagggggccgaggcgtcagcgtgggccccatcctcagcagcagtgcctcggatatcttctgcgacaatgagaatgggcctaacttccttttccacaaccggggcgatggcacctttgtggacgctgcggccagtgctggtgtggacgacccccaccagcatgggcgaggtgtcgccctggctgacttcaaccgtgatggcaaagtggacatcgtctatggcaactggaatggcccccaccgcctctatctgcaaatgagcacccatgggaaggtccgcttccgggacatcgcctcacccaagttctccatgccctcccctgtccgcacggtcatcaccgccgactttgacaatgaccaggagctggagatcttcttcaacaacattgcctaccgcagctcctcagccaaccgcctcttccgcgtcatccgtagagagcacggagaccccctcatcgaggagctcaatcccggcgacgccttggagcctgagggccggggcacagggggtgtggtgaccgacttcgacggagacgggatgctggacctcatcttgtcccatggagagtccatggctcagccgctgtccgtcttccggggcaatcagggcttcaacaacaactggctgcgagtggtgccacgcacccggtttggggcctttgccaggggagctaaggtcgtgctctacaccaagaagagtggggcccacctgaggatcatcgacgggggctcaggctacctgtgtgagatggagcccgtggcacactttggcctggggaaggatgaagccagcagtgtggaggtgacgtggccagatggcaagatggtgagccggaacgtggccagcggggagatgaactcagtgctggagatcctctacccccgggatgaggacacacttcaggacccagccccactggagtgtggccaaggattctcccagcaggaaaatggccattgcatggacaccaatgaatgcatccagttcccattcgtgtgccctcgagacaagcccgtatgtgtcaacacctatggaagctacaggtgccggaccaacaagaagtgcagtcggggctacgagcccaacgaggatggcacagcctgcgtggctcaagtggcctttttaggtgggtattcttcagccgcctctagaatctctgagcctctctctcgggcctcatatctttctctaggccttggactttgccttcagttatatgcactttaaatcccatcaataaaggaaaaaacaaaacaaaactaacagcctttgtggaaaactaaaaaaaaaaaaa

SEQ ID NO: 17 - DNA, 2089 bp, Homo sapiens
aagcagtggtaacaacgcagagtacgcgggggtataaagcgagcgcgctgaccccggcatgtccaggatgttaccgttcctgctgctgctctggtttctgcccatcactgaggggtcccagcgggctgaacccatgttcactgcagtcaccaactcagttctgcctcctgactatgacagtaatcccacccagctcaactatggtgtggcagttactgatgtggaccatgatggggactttgagatcgtcgtggcggggtacaatggacccaacctggttctgaagtatgaccgggcccagaagcggctggtgaacatcgcggtcgatgagcgcagctcaccctactacgcgctgcgggaccggcaggggaacgccatcggggtcacagcctgcgacatcgacggggacggccgggaggagatctacttcctcaacaccaataatgccttctcgggggtggccacgtacaccgacaagttgttcaagttccgcaataaccggtgggaagacatcctgagcgatgaggtcaacgtggcccgtggtgtggccagcctctttgccggacgctctgtggcctgtgtggacagaaagggctctggacgctactctatctacattgccaattacgcctacggtaatgtgggccctgatgccctcattgaaatggaccctgaggccagtgacctctcccggggcattctggcgctcagagatgtggctgctgaggctggggtcagcaaatatacagggggccgaggcgtcagcgtgggccccatcctcagcagcagtgcctcggatatcttctgcgacaatgagaatgggcctaacttccttttccacaaccggggcgatggcacctttgtggacgctgcggccagtgctggtgtggacgacccccaccagcatgggcgaggtgtcgccctggctgacttcaaccgtgatggcaaagtggacatcgtctatggcaactggaatggcccccaccgcctctatctgcaaatgagcacccatgggaaggtccgcttccgggacatcgcctcacccaagttctccatgccctcccctgtccgcacggtcatcaccgccgactttgacaatgaccaggagctggagatcttcttcaacaacattgcctaccgcagctcctcagccaaccgcctcttccgcgtcatccgtagagagcacggagaccccctcatcgaggagctcaatcccggcgacgccttggagcctgagggccggggcacagggggtgtggtgaccgacttcgacggagacgggatgctggacctcatcttgtcccatggagagtccatggctcagccgctgtccgtcttccggggcaatcagggcttcaacaacaactggctgcgagtggtgccacgcacccggtttggggcctttgccaggggagctaaggtcgtgctctacaccaagaagagtggggcccacctgaggatcatcgacgggggctcaggctacctgtgtgagatggagcccgtggcacactttggcctggggaaggatgaagccagcagtgtggaggtgacgtggccagatggcaagatggtgagccggaacgtggccagcggggagatgaactcagtgctggagatcctctacccccgggatgaggacacacttcaggacccagccccactggagtgtggccaaggattctcccagcaggaaaatggccattgcatggacaccaatgaatgcatccagttcccattcgtgtgccctcgagacaagcccgtatgtgtcaacacctatggaagctacaggtgccggaccaacaagaagtgcagtcggggctacgagcccaacgaggatggcacagcctgcgtgggctggtggagccctgtgttgaagatagtgacaccacaagttgggaagagccttggtccctgaatcactgaatcactgccttgaatcaccgcctggaatacctgttgatcaggaacacttacctggaacttcactgagcaggatacaaacttctattgtattaagctattaatacattaagatttgggggtgctaccttacataataaattcccatttcctcttgaaaaaaaaaaaa

SEQ ID NO: 18 - DNA, 2145 bp, Homo sapiens
aagcagtggtaacaacgcagagtacgcgggggtataaagcgagcgcgctgaccccggcatgtccaggatgttaccgttcctgctgctgctctggtttctgcccatcactgaggggtcccagcgggctgaacccatgttcactgcagtcaccaactcagttctgcctcctgactatgacagtaatcccacccagctcaactatggtgtggcagttactgatgtggaccatgatggggactttgagatcgtcgtggcggggtacaatggacccaacctggttctgaagtatgaccgggcccagaagcggctggtgaacatcgcggtcgatgagcgcagctcaccctactacgcgctgcgggaccggcaggggaacgccatcggggtcacagcctgcgacatcgacggggacggccgggaggagatctacttcctcaacaccaataatgccttctcgggggtggccacgtacaccgacaagttgttcaagttccgcaataaccggtgggaagacatcctgagcgatgaggtcaacgtggcccgtggtgtggccagcctctttgccggacgctctgtggcctgtgtggacagaaagggctctggacgctactctatctacattgccaattacgcctacggtaatgtgggccctgatgccctcattgaaatggaccctgaggccagtgacctctcccggggcattctggcgctcagagatgtggctgctgaggctggggtcagcaaatatacagggggccgaggcgtcagcgtgggccccatcctcagcagcagtgcctcggatatcttctgcgacaatgagaatgggcctaacttccttttccacaaccggggcgatggcacctttgtggacgctgcggccagtgctggtgtggacgacccccaccagcatgggcgaggtgtcgccctggctgacttcaaccgtgatggcaaagtggacatcgtctatggcaactggaatggcccccaccgcctctatctgcaaatgagcacccatgggaaggtccgcttccgggacatcgcctcacccaagttctccatgccctcccctgtccgcacggtcatcaccgccgactttgacaatgaccaggagctggagatcttcttcaacaacattgcctaccgcagctcctcagccaaccgcctcttccgcgtcatccgtagagagcacggagaccccctcatcgaggagctcaatcccggcgacgccttggagcctgagggccggggcacagggggtgtggtgaccgacttcgacggagacgggatgctggacctcatcttgtcccatggagagtccatggctcagccgctgtccgtcttccggggcaatcagggcttcaacaacaactggctgcgagtggtgccacgcacccggtttggggcctttgccaggggagctaaggtcgtgctctacaccaagaagagtggggcccacctgaggatcatcgacgggggctcaggctacctgtgtgagatggagcccgtggcacactttggcctggggaaggatgaagccagcagtgtggaggtgacgtggccagatggcaagatggtgagccggaacgtggccagcggggagatgaactcagtgctggagatcctctacccccgggatgaggacacacttcaggacccagccccactggagtgtggccaaggattctcccagcaggaaaatggccattgcatggacaccaatgaatgcatccagttcccattcgtgtgccctcgagacaagcccgtatgtgtcaacacctatggaagctacaggtgccggaccaacaagaagtgcagtcggggctacgagcccaacgaggatggcacagcctgcgtggaaagaaccttgctacttggtctctgcaatctccttggaaaatagaatgctcctgtagccagaacactctttggaaatgatgtaacgccacagaatatgctcacgaccagcctcagtagtgaaggcttgttataaaacatagatctattcttcatggggaaaaaaagtcatcccaggatataataaaattacacttaagaatgcagcaacccttttaaaaacatattcaacaaaaaaaagcccctgccgttttctgatatcatagtaatactcctaataaatcacaaaatatg

SEQ ID NO: 19 - protein, 637 aa, Homo sapiens
MSRMLPFLLLLWFLPITEGSQRAEPMFTAVTNSVLPPDYDSNPTQLNYGVAVTDVDHDGDFEIVVAGYNGPNLVLKYDRAQKRLVNIAVDERSSPYYALRDRQGNAIGVTACDIDGDGREEIYFLNTNNAFSGVATYTDKLFKFRNNRWEDILSDEVNVARGVASLFAGRSVACVDRKGSGRYSIYIANYAYGNVGPDALIEMDPEASDLSRGILALRDVAAEAGVSKYTGGRGVSVGPILSSSASDIFCDNENGPNFLFHNRGDGTFVDAAASAGVDDPHQHGRGVALADFNRDGKVDIVYGNWNGPHRLYLQMSTHGKVRFRDIASPKFSMPSPVRTVITADFDNDQELEIFFNNIAYRSSSANRLFRVIRREHGDPLIEELNPGDALEPEGRGTGGVVTDFDGDGMLDLILSHGESMAQPLSVFRGNQGFNNNWLRVVPRTRFGAFARGAKVVLYTKKSGAHLRIIDGGSGYLCEMEPVAHFGLGKDEASSVEVTWPDGKMVSRNVASGEMNSVLEILYPRDEDTLQDPAPLECGQGFSQQENGHCMDTNECIQFPFVCPRDKPVCVNTYGSYRCRTNKKCSRGYEPNEDGTACVAQVAFLGGYSSAASRISEPLSRASYLSLGLGLCLQLYAL

SEQ ID NO: 20 - protein, 618 aa, Homo sapiens
MSRMLPFLLLLWFLPITEGSQRAEPMFTAVTNSVLPPDYDSNPTQLNYGVAVTDVDHDGDFEIVVAGYNGPNLVLKYDRAQKRLVNIAVDERSSPYYALRDRQGNAIGVTACDIDGDGREEIYFLNTNNAFSGVATYTDKLFKFRNNRWEDILSDEVNVARGVASLFAGRSVACVDRKGSGRYSIYIANYAYGNVGPDALIEMDPEASDLSRGILALRDVAAEAGVSKYTGGRGVSVGPILSSSASDIFCDNENGPNFLFHNRGDGTFVDAAASAGVDDPHQHGRGVALADFNRDGKVDIVYGNWNGPHRLYLQMSTHGKVRFRDIASPKFSMPSPVRTVITADFDNDQELEIFFNNIAYRSSSANRLFRVIRREHGDPLIEELNPGDALEPEGRGTGGVVTDFDGDGMLDLILSHGESMAQPLSVFRGNQGFNNNWLRVVPRTRFGAFARGAKVVLYTKKSGAHLRIIDGGSGYLCEMEPVAHFGLGKDEASSVEVTWPDGKMVSRNVASGEMNSVLEILYPRDEDTLQDPAPLECGQGFSQQENGHCMDTNECIQFPFVCPRDKPVCVNTYGSYRCRTNKKCSRGYEPNEDGTACVGWWSPVLKIVTPQVGKSLGP

SEQ ID NO: 21 - protein, 612 aa, Homo sapiens
MSRMLPFLLLLWFLPITEGSQRAEPMFTAVTNSVLPPDYDSNPTQLNYGVAVTDVDHDGDFEIVVAGYNGPNLVLKYDRAQKRLVNIAVDERSSPYYALRDRQGNAIGVTACDIDGDGREEIYFLNTNNAFSGVATYTDKLFKFRNNRWEDILSDEVNVARGVASLFAGRSVACVDRKGSGRYSIYIANYAYGNVGPDALIEMDPEASDLSRGILALRDVAAEAGVSKYTGGRGVSVGPILSSSASDIFCDNENGPNFLFHNRGDGTFVDAAASAGVDDPHQHGRGVALADFNRDGKVDIVYGNWNGPHRLYLQMSTHGKVRFRDIASPKFSMPSPVRTVITADFDNDQELEIFFNNIAYRSSSANRLFRVIRREHGDPLIEELNPGDALEPEGRGTGGVVTDFDGDGMLDLILSHGESMAQPLSVFRGNQGFNNNWLRVVPRTRFGAFARGAKVVLYTKKSGAHLRIIDGGSGYLCEMEPVAHFGLGKDEASSVEVTWPDGKMVSRNVASGEMNSVLEILYPRDEDTLQDPAPLECGQGFSQQENGHCMDTNECIQFPFVCPRDKPVCVNTYGSYRCRTNKKCSRGYEPNEDGTACVERTLLLGLCNLLGK

(19) United States
(12) Patent Application Publication
(10) Pub. No.: US 2004/0072286 A1
(43) Pub. Date: Apr. 15, 2004

(54) Extracellular matrix protein

(76) Inventors: Hendrik Cornelis Heus, Rueil Malmaison (FR); Robert Louis Hubert Nelisssen, Oss (NL); Cornelis Maria Leonardus Meeuwisse, Eindhoven (NL)

Correspondence Address: William M Blackstone, Akzo Nobel Intervet Inc, Patent Department, 405 State Street, Millsboro, DE, 19966, US

(21) Appl. No.: 10/332,929

(22) PCT Filed: Jul. 9, 2001
(86) PCT No.: PCT/EP01/07888

(30) Foreign Application Priority Data
Jul. 13, 2000 (EP) 00202495.8

(51) Int. Cl. A61K 38/17; C12P 21/02; C12N 5/06; C07K 14/47

(52) U.S. Cl. 435/69.1; 435/320.1; 435/325; 514/12; 530/350; 536/23.5

(57) ABSTRACT

The invention describes a novel protein and variants thereof as well as the genes coding for these proteins. The protein is useful in the prevention of inflammatory diseases, more specifically in the induction of T cell tolerance to the protein in patients suffering from rheumatoid arthritis.

The current invention relates to the isolation of a new polynucleotide molecule encoding a novel extracellular matrix protein, i.e. SCIM-1. The encoded protein or derivatives thereof, are useful for diagnosing, preventing or treating medical conditions. They can be used as modulatory agents for the treatment of autoimmune diseases, and more specifically rheumatoid arthritis.

The primary functional role of the immune system is to protect the individual against invading pathogens bearing foreign, that is non-self, antigens. In order to fulfil this function in a safe and effective manner, a mechanism is required to discriminate between foreign antigens and autoantigens derived from the individuals own body. Failure of this process of self-non-self discrimination, that is loss of immune tolerance to self-antigens, may lead to immune reactivity to autoantigens resulting in autoimmune disease, involving tissue damage and loss of organ function.

Autoimmune diseases are a major problem in human health care. Some autoimmune diseases may be the result of an immunological process directed at one antigen or antigenic complex whereas in others the autoimmune reaction may involve many types of antigens that may be present in multiple organs. Several lines of evidence have indicated that the immune system is involved in the pathology of autoimmune diseases. First, the chances of individuals to develop an autoimmune disease are closely linked to their genetic backgrounds: genes encoding major histocompatibility complex (MHC) class II molecules that present (auto)antigens to responding T cells which recognise MHC-peptide complexes show a strong genetic linkage to disease susceptibility. Second, cells of the immune system such as monocyte/macrophages and T cells infiltrate target organs. Third, T cells of patients with autoimmune diseases proliferate in vitro in response to potentially involved autoantigens. Fourth, studies in animal models of autoimmunity have unequivocally demonstrated that cells of the immune system such as monocyte/macrophages and T cells are involved in induction and expression of disease activity.

A disease as rheumatoid arthritis (RA) can illustrate the immunopathology that may occur in case of an autoimmune disease. RA presents itself as a chronic multisystem disease in which the common clinical manifestation is the persistent inflammatory synovitis accompanied by proliferation of synovial cells, pannus formation, cartilage degradation and bone erosion, and ultimately joint deformity resulting in loss of function.

Existing therapies for the treatment of autoimmune disorders, such as RA, in which the immune system generates an unwanted and undesirable inflammatory response, are inadequate. Treatment has focused on relief of symptoms of autoimmune disease rather that on its cause. Most drugs used in the treatment of autoimmune diseases, e.g. steroids and non-steroidal anti-inflammatory compounds, are non-specific and have significant toxic side effects. This is especially problematic since autoimmune diseases are chronic conditions, which require the prolonged administration of drugs.

Antigen-driven, non-toxic immunomodulation therapy provides a very attractive alternative for the non-specific immunosuppression. This antigen-specific therapy involves the treatment of patients with the target (auto)antigen or with synthetic T cell-reactive peptides derived from the (auto)antigen. These synthetic peptides correspond to T cell epitopes of the (auto)antigen and can be used to induce specific T cell tolerance both to themselves and to the (auto)antigen. The controlled administration of the target (auto)antigen can be very effective in desensitisation of the immune system. Desensitisation or immune tolerance of the immune system is based on the long-observed phenomenon that animals which have been fed or have inhaled an antigen or epitope are less capable of developing a systemic immune response towards said antigen or epitope when said antigen or epitope is introduced via a systemic route.

With regard to the use of an antigen for immune therapy, the human cartilage (IC) gp-39 protein has very recently been reported to be effective in induction of systemic immune tolerance (Joosten et al. Arthritis Rheum. 43:645-655, 2000) for the treatment of arthritis. Upon intranasal administration HC gp-39 interfered with development of collagen type II-induced arthritis in mice. Both disease activity and joint destruction ameliorated as a result of the treatment.

In a search for proteins that are relevant for autoimmune diseases such as rheumatoid arthritis, we have identified a novel gene SCIM-1 (Synovium/Cartilage Inflammation-linked Messenger 1). The protein that is encoded by the SCIM-1 mRNA has not been reported earlier. Upon identification of the SCIM-1 mRNA it was established that only fragments of the mRNA were present in public domain databases as expressed sequence tags (ESTs). From the public ESTs it was not possible to reconstruct the identified SCIM-1 cDNA.

The best homology with the SCIM-1 protein was found with the EGF-like domains of fibulin-1, an extracellular protein containing 9 EGF-like domains. Fibulin-1 can bridge the extracellular matrix of the vessel wall with integrin-αIIbβ3 on platelets, via an interaction with fibrinogen (Godyna et al. Blood 88:2569-2577,1996). Via its EGF-like domains fibulin-1 binds to nidogen, fibronectin, laminin, fibrinogen, NOVH, aggrecan, and versican (Tran et al. J. Biol. Chem. 272:22600-22606, 1997; Perbal et al. Proc. Natl. Acad. Sci. USA 96:869-874, 1999; Barth et al. Matrix Biol. 17:635-646, 1998). High expression of fibulin-1 seems to interfere with tumor formation and invasion (Qing et al. Oncogene 15:2159-2168, 1997). It is conceivable that SCIM-1 has similar capabilities, involving its RGD-motif en EGF-like domain.

Based on the report of Sarkissian and Lafyatis (J. Immunol. 162:1772-1779, 1999), the putative interaction of SCIM-1 with integrins could be relevant for RA, since proliferation of fibroblast-like synoviocytes (FLS), e.g. from RA patients, required a signal provided by integrin-binding extracellular matrix proteins in addition to a mitogenic cytokine, e.g. PDGF. Wang et al. (Arthritis Rheum. 40:1298-1307, 1997) suggested that αv, α4, and α5 integrins are required for IL-1β-stimulated invasion of articular cartilage by RA-FLS.

The SCIM-1 gene appeared to be expressed relatively high in the synovial and cartilage tissues. Based on a partial cDNA sequence, a full-length cDNA was isolated from human chondrocytes. Probing multiple tissue northern blots, it was shown that the SCIM-1 gene was expressed in primary chondrocytes from healthy human donors, while some expression was also observed in brain and lung, although the SCIM-1 mRNAs in these tissues seemed to differ in length. Only low or no expression was observed in other human tissues such as heart, placenta, liver, skeletal muscle, spleen, kidney and colon or cell lines of monocytes, arterial endothelial cells, and (cultured) synovial fibroblasts.

SCIM-1 expression appears to be positively correlated to a differentiated chondrocyte phenotype, since expression was only detected in primary chondrocytes but not in human primary chondrocytes transformed with SV40. Furthermore, it was shown that expression of the SCIM-1 gene in human chondrocytes was enhanced in the presence of the RA-related cytokines TNFα and ILβ. In chondrocytes, transcription of the SCIM-1 gene results in a 2.7 kb mRNA which encodes a protein of 653 amino acids. Based on the multiple tissue Northern blot data and the sequences of Incyte and public domain databases, It was found that the SCIM-1 transcript undergoes a different splicing in tissues of the joint, brain, and lungs.

Based on these predicted protein motifs the SCIM-1 protein is expected to be an extracellular matrix protein or a protein that is localized on the cell surface of chondrocytes and synoviocytes and possibly some other cell types.

The main problem in (auto)immune diseases (such as e.g. RA) is that the precise targets or antigens that the immune system is adversely reacting to are largely unknown, implicating that modulating a disease entity in an antigen-specific fashion may not be possible.

It would be an important advantage, however, if an antigen-driven, non-toxic form of immunomodulation therapy could be utilised without knowledge of the antigen(s) that are involved as a target in the (auto)immune response. Such an antigen-driven therapy would involve the generation of antigen-specific modulator cells with the use of an antigen that is expected to be released or produced during the autoimmune process. Such an antigen would become available during inflammation or tissue destruction. In case of an autoimmune disease, the locally produced autoantigen should then activate or reactivate modulator cells induced with such an antigen.

To effectively use tolerance induction therapy to treat T cell mediated cartilage destruction, there is a great need to identify T cell-reactive (poly)peptides which can desensitise patients against the autoantigen that is activating the T cells responsible for the inflammatory process.

It is an object of the invention to provide a (poly)peptide which is capable of inducing systemic immune tolerance, more in particular specific T cell tolerance, preferably to the responsible cartilage antigen in patients suffering from T cell-mediated cartilage destruction. It has been found that SCIM-1 fulfils the above mentioned requirements and can be used as an effective toleragen in modulating the immune system.

In the present invention under induction of systemic immune tolerance is to be understood the stimulation of antigen specific lymphocytes by antigen presenting cells (APC) in such a way that the lymphocytes acquire a state in which they produce anti-inflammatory cytokines. Anti-inflammatory cytokines may for example be IL-4, IL-10, and/or TGF-β. Lymphocytes brought to tolerance by APC are able to impose their anti-inflammatory state to other sites of the body, e.g. sites of ongoing inflammation.

The immune system protects individuals against foreign antigens and responds to exposure to a foreign antigen by activating specific cells such as T- and B-lymphocytes and producing soluble factors like interleukins, antibodies and complement factors. The antigen to which the immune system responds is degraded by the antigen presenting cells (APCs) and a fragment of the antigen is expressed on the cell surface associated with a major histocompatibility complex (NC) class II glycoprotein. The MHC-glycoprotein-antigen-fragment complex is presented to a T cell, which by virtue of its T cell receptor recognises the antigen fragment conjointly with the MHC class II protein to which it is bound. The T cell becomes activated, i.e. proliferates and/or produces interleukins, resulting in the expansion of the activated lymphocytes directed to the antigen under attack (Grey et al., Sci. Am., 261:38-46, 1989).

Self-antigens are also continuously processed and presented as antigen fragments by the MHC glycoproteins to T cells (Jardetsky et al., Nature 353:326-329, 1991). Self recognition thus is intrinsic to the immune system. Under normal circumstances the immune system is tolerant to self-antigens and activation of the immune response by these self-antigens is avoided. When tolerance to self-antigens is lost, the immune system becomes activated against one or more self-antigens, resulting in the activation of autoreactive T cells and sometimes also the production of autoantibodies. This phenomenon is referred to as autoimmunity. As the immune response in general is destructive, i.e. meant to destroy the invasive foreign antigen, autoimmune responses can cause destruction of the body's own tissue.

It will thus be clear that fragments of the SCIM-1 protein will be expressed by the APC and that therefore also fragments of the SCIM-1 protein are capable of evoking an immune response. Also proteins of other species having a similar function or at least being structurally closely related to the human SCIM-1 protein might perform the same toleragenic effect. Thus, also homologous polypeptides or parts thereof are included in the invention.

The proteins according to the present invention include the polypeptide comprising SEQ ID NO: 2 but also polypeptides with a similarity of 70%, preferably 90%, more preferably 95%, 98%, most preferably 99%. Also portions of such polypeptides still capable of conferring the toleragenic effects are included. Such portions may be finctionial per se, e.g. in solubilized form or they might be linked to other polypeptides, either by known biotechnological ways or by chemical synthesis, to obtain chimeric proteins.

As used herein the term similarity is as defined in NCBI-BLAST 2.0.10 [Aug. 26, 1999] (Altschul et al., Nucleic Acids Res. 25:3389-3402, 1997).

The fragments of the SCIM-1 protein or homologous polypeptides are to be understood subsequences of the protein. These subsequences can modulate lymphocyte functioning. Preferably they have the following functional immunomodulating characteristics: i) peptides can be bound by the disease-associated MHC molecules, preferably HLA-DRB1*0101, DRB1*0401, DRB1*0404, DRB1*0408, DRB1*0405, DQB*0301, or DQB*0302, and ii) peptides must be able to provoke a T cell response in humans, preferably autoimmune patients, more preferably RA patients. Such a response can for example be measured in an in vitro T cell proliferation assay or in an assay for the detection of T cell cytokine production (e.g. ELISA or ELISPOT) (Coligan et al., Current Protocols in Immunology. John Wiley & Sons, Inc., 1998). Preferably the peptides must also be recognized by T cells in animals transgenic for the relevant human MHC class II molecules, as mentioned above, and human CD4 upon immunization with a SCIM-1 (poly)peptide.

The length of these sub-sequences is not important provided that it comprises the epitope to be recognized by the relevant MHC molecule. Preferably these peptides have an amino acid sequence of 9-55 amino acid residues. More preferably the peptides as have an amino acid sequence of 9-35, in particular 9-25 amino acid residues. Much more preferred are peptides having an amino acid sequence of 9-15 amino acid residues. Highly preferred are peptides having an amino acid sequence of 13 or 14 amino acid residues.

Variations that can occur in a sequence, especially of smaller peptides, may be demonstrated by (an) amino acid difference(s) in the overall sequence or by deletions, substitutions, insertions, inversions or additions of (an) amino acid(s) in said sequence. Amino acid substitutions that are expected not to essentially alter biological and immunological activities have been described. Amino acid replacements between related amino acids or replacements which have occurred frequently in evolution are, inter alia Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Based on this information Lipman and Pearson developed a method for rapid and sensitive protein comparison (Science, 227:1435-1441, 1985) and determining the functional similarity between homologous polypeptides.

Also within the scope of the invention are multimers of the peptides such as for example a dimer or trimer of the peptides according to the invention. A multimer according to the invention can either be a homomer, consisting of a multitude of the same peptide, or a heteromer consisting of different peptides.

It will be clear to those skilled in the art that the (poly)peptides may be extended at either side of the peptide or at both sides and still exert the same immunological function. The extended part may be an amino acid sequence similar to the natural sequence of the protein. However, the (poly)peptide might also be extended by non-natural sequences. It will be clear that the (poly)peptide need not to exert its original function and as such might be inactive while still performing its immunological function according to the invention. The (poly)peptide according to the invention might be connected to MHC molecules, such that the binding groove is occupied by the peptide. A flexible linker molecule, preferably also consisting of amino acid sequences might connect the peptide. The MHC molecules need not to possess their constant domains and might consist of their variable domains only, either directly connected to each other or connected through a flexible linker. The advantage of such a complex is that it might exist in a soluble form and can directly be recognised by T cells.

Thus, according to the invention the (poly)peptides, said (poly)peptides resembling the MHC Class II restricted T-cell epitopes present on the antigen comprising the polypeptide of SEQ ID NO: 2 or fragments thereof comprising these epitopes are very suitable for use in a therapy to induce systemic immune tolerance to said antigen in mammals, more specifically humans, suffering from lymphocyte or T-cell mediated cartilage destruction, such as for example arthritis, more specifically rheumatoid arthritis. Optionally such a treatment can be combined with the administration of other medicaments such as DMARDs (Disease Modifying Anti-Rheumatic Drugs e.g. sulfasalazine, anti-malarials (chloroquine, hydroxychloroquine) injectable or oral gold, methotrexate, D-penicillamine, azathioprine, cyclosporine, mycophenolate), NSAIDs (non steroidal anti inflammatory drugs), corticosteroids or other drugs known to influence the course of the disease in autoimmune patients.

The polypeptides according to the invention can also be used to modulate lymphocytes that are reactive to antigens other than said antigen but are present in the same tissue as the antigen i.e. proteins or parts thereof comprising the polypeptide according to SEQ ID NO: 2. By the induction of antigen-specific T-cell tolerance, autoimmune disorders can be treated by systemic immune tolerance. More in general, the cells to be modulated are hematopoietic cells. In general, in order to function as a toleragen the peptide must fulfil at least two conditions i.e. it must possess an immune modulating capacity and it must be expressed locally usually as part of a larger protein.

Thus, the present invention provides a method to treat patients suffering from inflammatory autoimmune diseases, by administration of a pharmaceutical preparation comprising the (poly)peptide according to the invention. The (poly)peptide comprises T-cell epitopes, which are recognised by and are able to stimulate autoreactive T-cells.

These T cells may be found e.g. in the blood of patients suffering from inflammatory disorders. Such patients may suffer from diseases like Graves' diseases, juvenile arthritis, primary glomerulonephritis, polyarthritis, osteoarthritis, Sjögren's syndrome, myasthenia gravis, rheumatoid arthritis, Addison's disease, primary biliary sclerosis, uveitis, systemic lupus erythematosis, inflammatory bowel disease, multiple sclerosis or diabetes. The polypeptides according to the present invention therefore can be used in the preparation of a pharmaceutical to prevent inflammatory diseases. Administration of SCIM-1 will induce systemic immune tolerance. More specifically the polypeptides can be used in the preparation of a pharmaceutical to induce specific immune tolerance in patients suffering from inflammatory diseases, preferably immune-cell mediated cartilage destruction. The immune cell, preferably is a T cell. The most preferred disease is arthritis, more preferably rheumatoid arthritis.

Treatment of autoimmune disorders with the peptides according to the invention makes use of the fact that systemic immune tolerance is induced to unrelated but co-localised antigens. The regulatory cells secrete in an antigen specific fashion pleiotropic proteins such as cytokines which may downmodulate the immune response.

The polypeptides according to the invention can be prepared by recombinant DNA techniques.

The present invention provides for such a DNA sequence encoding a protein or polypeptide according to the invention.

The invention also includes the entire mRNA sequence part of which is indicated in SEQ ID NO: 1. A complete coding DNA sequence is shown in SEQ ID NO: 1 nucleotides 59-2017. Furthermore, to accommodate codon variability, the invention also includes sequences coding for the same amino acid sequences as the sequences disclosed herein. Also portions of the coding sequences coding for individual polypeptides having the same immunological function are part of the invention as well as allelic and species variations thereof. Sometimes, a gene is expressed in a certain tissue as a splicing variant, resulting in an altered 5′ or 3′ mRNA or the inclusion of an additional exon sequence. These sequences as well as the proteins encoded by these sequences all are expected to perform the same or similar functions and form also part of the invention.

In particular, SEQ ID NO: 16, SEQ ID NO: 17 and SEQ ID NO: 18 represent specific splice variants which differ from SEQ ID NO: 1 in the sequence downstream of nucleotide 1852 of SEQ ID NO: 1. Translation of this splice variants leads to a truncated version of the protein in SEQ ID NO: 2, as shown in SEQ ID NO: 19, SEQ ID NO: 20 and SEQ ID NO: 21, respectively. The coding sequences of these latter nucleic acids run from positions 59-1969, 59-1912 and 59-1894, respectively and have the first 598 amino acids of SEQ ID NO: 2 in common. The complete proteins preferably are mature proteins. The signal sequence most likely is 19 amino acids but the length may differ slightly.

The sequence information as provided herein should not be so narrowly construed as to require inclusion of erroneously identified bases. The specific sequence disclosed herein can be readily used to isolate the complete genes which in turn can easily be subjected to further sequence analyses thereby identifying sequencing errors.

Thus, the present invention provides also for isolated polynucleotides encoding SCIM-1, truncated versions or fragments thereof.

The DNA according to the invention may be obtained from cDNA. The tissues preferably are from human origin. Preferably ribonucleic acids are isolated from fetal brain, fetal liver, fetal spleen, placenta or other tissues. Alternatively, the coding sequence might be genomic DNA, or prepared using DNA synthesis techniques. The polynucleotide may also be in the form of RNA. If the polynucleotide is DNA, it may be in single stranded or double stranded form. The single strand might be the coding strand or the non-coding (anti-sense) strand.

The present invention further relates to polynucleotides having slight variations or have polymorphic sites. Polynucleotides having slight variations encode polypeptides which retain the same biological function or activity as the natural, mature protein. Polymorpic sites are useful for diagnostic purposes. Such polynucleotides can be identified by hybridization under preferably highly stringent conditions. According to the present invention the term “stringent” means washing conditions of 1×SSC, 0.1% SDS at a temperature of 65° C.; highly stringent conditions refer to a reduction in SSC towards 0.3×SSC, more preferably to 0.1×SSC. Preferably the first two washings are subsequently carried out twice each during 15-30 minutes. If there is a need to wash under highly stringent conditions an additional wash with 0.1×SSC is performed once during 15 minutes. Hybridization can be performed e.g. overnight in 0,5M phosphate buffer pH7.5/7% SDS at 65° C.

Alternatively, also fragments of the above mentioned polynucleotides which code for polypeptides having the same immunological function are embodied in the invention.

Also the encoded proteins or polypeptides derived from the proteins form part of the invention.

A nucleic acid sequence coding for the protein, a peptide according to the invention, a multimer of said peptides or a chimeric peptide is inserted into an expression vector. Suitable expression vectors comprise the necessary control regions for replication and expression. The expression vector can be brought to expression in a host cell. Suitable host cells are, for instance, bacteria, yeast cells and mammalian cells. Such techniques are well known in the art, see for instance Sambrook et al., Molecular Cloning: a Laboratory Manual, Cold Spring Harbor laboratory Press, Cold Spring Harbor, 1989.

The (smaller) (poly)peptides according to the invention can also be prepared by well known organic chemical methods for peptide synthesis such as, for example, solid-phase peptide synthesis described for instance in J. Amer. Chem. Soc. 85:2149 (1963) and Int. J. Peptide Protein Res. 35:161-214 (1990).

The (poly) peptides may be stabilised by C- and/or N-terminal modifications, which will decrease exopeptidase catalysed hydrolysis. The modifications may include: C-terminal acylation, (e.g. acylation=Ac-peptide), N-terminal amide introduction, (e.g. peptide-NH₂) combinations of acylation and amide introduction (e.g. Ac-peptide-NH₂) and introduction of D-amino acids instead of L-amino acids (Powell et al., J. Pharm. Sci., 81:731-735, 1992).

Other modifications are focussed on the prevention of hydrolysis by endopeptidases. Examples of these modifications are: introduction of D-amino acids instead of L-amino acids, modified amino acids, cyclisation within the peptide, introduction of modified peptide bonds, e.g. reduced peptide bonds ψ[CH₂NH] and e.g. peptoids (N-alkylated glycine derivatives) (Adang et al., Recl. Trav. Chim. Pays-Bas, 113:63-78, 1994 and Simon et al., Proc. Natl. Acad. Sci. USA, 89:9367-9371, 1992).

The tolerogenic peptides according to the invention can be identified by using a method comprising the steps of

a) introducing into a suitable host cell a DNA fragment encoding SCIM-1 peptide fragments;

b) culturing the host cells under conditions to allow expression of the introduced DNA sequence;

c) bringing the expression product in contact with lymphocytes; and

d) establishing the lymphocyte activity.

Preferably, the expression product is contacted with lymphocytes in vivo e.g. by administration of the product to animals. The lymphocyte activity can e.g. be measured by determination of the anti-inflammatory cytokines.

Alternatively, the expression product of the host cells under step b might be isolated, brought subsequently into contact with antigen presenting cells and T cells and the T cell activity might be established.

It will be clear that in the above described screening methods the peptides to be tested might also be synthesized chemically.

The peptides thus identified can be used for the formulation of a pharmaceutical composition comprising mixing the peptide with a pharmaceutically acceptable carrier.

According to the invention, patients suffering from T-cell mediated destruction of the articular cartilage can be treated with a therapeutical composition comprising one or more peptides according to the invention and a pharmaceutical acceptable carrier. Administration of the pharmaceutical composition according to the invention will induce systemic immune tolerance, in particular tolerance of the specific autoreactive T cells of these patients, to the autoantigenic proteins in the articular cartilage under attack and other self antigens which display the identified MHC Class II binding T cell epitopes characterised or mimicked by the amino acid sequences of one or more of the peptides according to the invention. The induced tolerance thus will lead to a reduction of the local inflammatory response in the articular cartilage under attack.

The (poly)peptides according to the invention have the advantage that they have a specific effect on the autoreactive T cells thus leaving the other components of the immune system intact as compared to the non-specific suppressive effect of immunosuppressive drugs.

Systemic immune tolerance can be attained by administering high or low doses of peptides according to the invention. The amount of peptide will depend on the route of administration, the time of administration, the age of the patient as well as general health conditions and diet.

In general, a dosage of 0.01 to 10000 μg of peptide per kg body weight, preferably 0.05 to 500 μg, more preferably 0.1 to 100 μg of peptide can be used.

Pharmaceutical acceptable carriers are well known to those skilled in the art and include, for example, sterile saline, lactose, sucrose, calcium phosphate, gelatin, dextrin, agar, pectin, peanut oil, olive oil, sesame oil and water. Other carriers may be, for example MHC class II molecules, if desired embedded in liposomes.

In addition the pharmaceutical composition according to the invention may comprise one or more adjuvants. Suitable adjuvants include, amongst others, aluminium hydroxide, aluminium phosphate, amphigen, tocophenols, monophosphenyl lipid A, muramyl dipeptide and saponins such as Quill A. Preferably, the adjuvants to be used in the tolerance therapy according to the invention are mucosal adjuvants such as the cholera toxin B-subunit or carbomers, which bind to the mucosal epithelium. The amount of adjuvant depends on the nature of the adjuvant itself.

Furthermore the pharmaceutical composition according to the invention may comprise one or more stabilisers such as, for example, carbohydrates including sorbitol, mannitol, starch, sucrosedextrin and glucose, proteins such as albumin or casein, and buffers like alkaline phosphates.

Suitable administration routes are e.g. intramuscular injections, subcutaneous injections, intravenous injections or intraperitoneal injections, oral administration and nasal administration such as sprays.

For testing the ability of the (poly)peptides to modulate (auto)immune responses several murine models have been shown to be suitable, such as collagen induced arthritis in mice (CIA), adjuvant arthritis in rats, experimental allergic encephalomyelitis in mice and non-obese diabetes in mice (NOD) or in transgenic mice. Antigen may be administered intravenously, intraperitoneally, orally or nasally in such models (review by Liblau et al., Immunol. Today 18:599-603, 1997). To facilitate the read-out in these models, it is of importance to increase the confidence interval. According to the present invention it has been found that incidence and clinical score in arthritis models can be improved by combining the original trigger of arthritis, e.g. collagen type II in CIA with a peptide derived from the extracellular matrix protein aggrecan. This peptide might preferably be administered simultaneously with the original trigger although a separate administration might also be possible.

SCIM-1 or the peptides according to the invention are also very suitable for use in a diagnostic method to detect the presence of activated autoreactive T cells involved in the chronic inflammation of the articular cartilage.

The diagnostic method according to the invention comprises the following steps:

a) isolation of the peripheral blood mononuclear cells (PBMC) from a blood sample of an individual,

b) culture said PBMC under suitable conditions,

c) incubation of said PBMC culture in the presence of the autoantigen or one or more peptides derived thereof according to the invention, and

d) detection of a response of T cells, for example a proliferative response, indicating the presence of activated autoreactive T cells in the individual.

In case of detection of a response by measuring the proliferative response of the autoreactive T cells, the incorporation of a radioisotope such as for example 3H-thymidine is a measure for the proliferation. A response of the autoreactive T cells present in the PBMC can also be detected by measuring the cytokine release with cytokine-specific ELISA, or the cytotoxicity with ⁵¹Chromium release. Another detection method is the measurement of expression of activation markers by FACS analysis, for example of II-2R. A diagnostic composition comprising one or more of the peptides according to the invention and a suitable detecting agent thus forms part of the invention. Depending on the type of detection, the detection agent can be a radioisotope, an enzyme, or antibodies specific for cell surface or activation markers.

Also within the scope of the invention are test kits which comprise one or more peptides according to the invention. These test kits are suitable for use in a diagnostic method according to the invention.

Thus, the present invention provides for a method to detect whether autoaggressive T cells reactive towards SCIM-1 are present in patients suffering from T-cell mediated cartilage destruction such as for example arthritis, in particular rheumatoid arthritis. If SCIM-1-specific T cells are present, tolerization of these T cells with a pharmaceutical compostion comprising SCIM-1 or peptides according to the present invention or combinations thereof can delay or suppress arthritis development.

The following examples are illustrative for the invention and should in no way be interpreted as limiting the scope of the invention.

### LEGENDS TO THE FIGURES

FIG. 1

RT-PCR on human cartilage cDNA, using oligonucleotides specific for SCIM-1 and GAPDH. Detection of SCIM-1 gene expression is shown for healthy cartilage (knee, male; traffic accident; lane 1), and cartilage from 4 RA patients (knee/hip, 3 female and 1 male; lanes 2-5). Detection of GAPDH gene expression for the same 4 RA samples is shown in lanes 7-10. As a DNA fragment length marker the 100 bp-ladder (Gibco-BRL) was run in parallel in lane 6 (the major band in the middle of the lane corresponds to 600 bp).

FIG. 2

SCIM-1 probe (nt560-1048 of SEQ ID NO: 1) hybridized on custom-made joint Northern blot (upper panel). Represented human cells/tissues: U937 monocytes+PMA-ionomycin (lane 1), HAEC endothelial cells±TNFα (lanes 2-3, respectively), SCRO14.SF primary synovial fibroblasts+TNFα (lane 4), PCG SV40.04 transformed chondrocytes±TNFA (lanes 5-6, respectively), and primary chondrocytes±TNFα (lanes 7-8, respectively). Per lane 1-2 ug of poly(A+) RNA was loaded. As a control the blot was probed with a ³²P-labelled cDNA derived from the human β-actin gene (lower panel).

FIG. 3

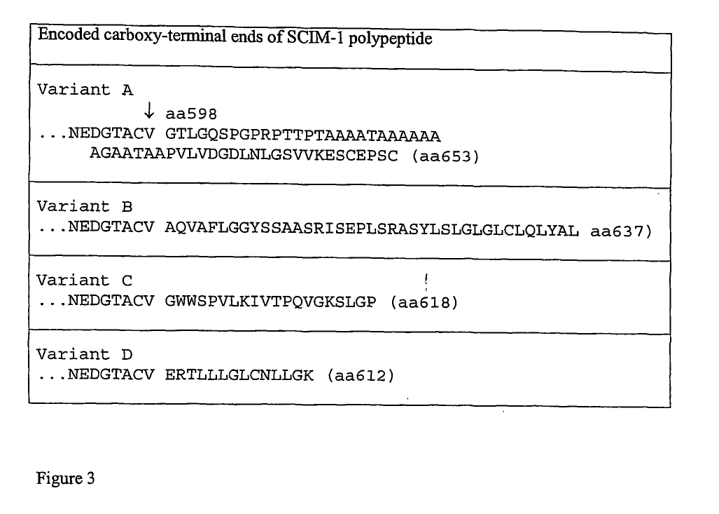

Alternative carboxy terminal ends as encoded by 4 different SCIM-1 mRNA species, i.e. variants A-D. The encoded C-termini are indicated and amino acids are shown in one-letter code. Sequences are different as from amino acid 599. Encoded amino acid sequences of variants A-D are represented by SEQ ID NO: 20-23, respectively, and the corresponding nucleic acids are represented by SEQ ID NO: 16-19, respectively.

FIG. 4

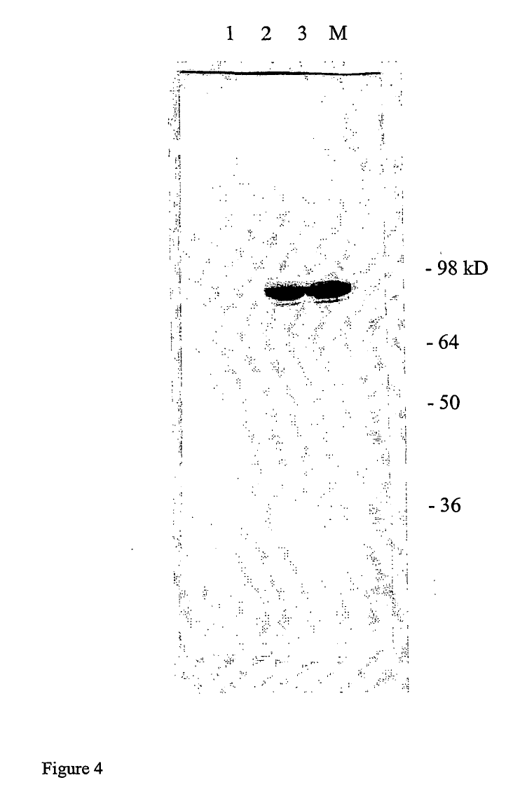

Detection of SCIM-1 variant A/His6 expression in transfected CHO cells. Samples of culture supernatant of CHO cells transfected with pNGVI (lane 1) or pNGV1.SCIM1.variantA(His6) (lanes 2 and 3) were separated on an SDS-polyacrylamide gel and subsequently Western blotted. Proteins containing a (His6)-tail were detected using an anti(His6)-tag monoclonal antibody (Dianova GMBH). A protein molecular weight marker was run in lane 4 (M).

### EXAMPLES

### Example 1

Identification of the SCIM-1 Gene

The cDNA libraries that were available from the LifeSeq database (Incyte Pharmaceuticals) were clustered based on their gene content in order to define custom tissue categories that represent RA relevant tissues such as joint tissues. Distances between cDNA libraries were calculated using the squared Euclidian distance measurement, and the results were clustered into a tree using an unweighted average linkage method (Fry 1993, Biological Data Analysis, Oxford University Press, New York). Two of the resulting clusters contained cDNAs of 5 libraries each derived from non-tumor synovial or cartilage tissues. For each of the synovial cDNA libaries data of 4038 to 5627 sequence reactions were available, and 1554 to 7231 for each of cartilage cDNA libraries. These sequence data were probed for genes of which expression seemed to be relatively restricted to these clusters. Two partial cDNA fragments, each composed of overlapping Incyte-est sequences and parts of SEQ ID NO: 1, were identified as positive hits. Since their expression patterns appeared to be similar, it was assumed that they were derived from the same gene.

### Example 2

SCIM-1 Gene expression in RA Cartilage.

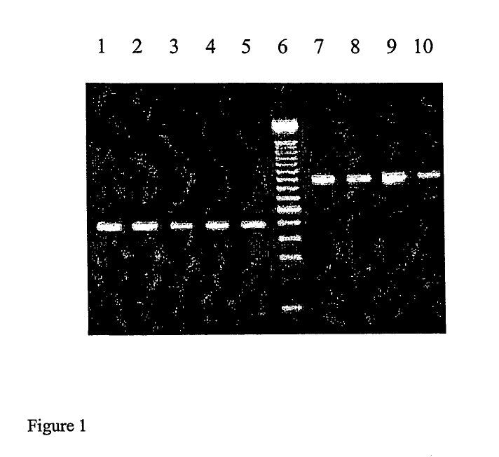

Expression of the SCIM-1 gene in diseased tissue was detected via RT-PCR with SCIM-1-specific oligonucleotides (SEQ ID NO: 3,5′TTGCCAATTACGCCTACGGT and SEQ ID NO: 4, 5′CCTGGTCATTGTCAAAGTCGG) on cDNA that was derived from cartilage samples of 4 RA patients and 1 healthy donor. The arthritic cartilage was obtained during joint replacement surgery of the knee. Chondrocytes were isolated enzymatically from the cartilage (Cornelissen et al., 1993, J. Tiss. Cult. Meth. 15:139-146) upon which RNA was isolated using Trizol (Gibco-BRL) or RNAzol B (Campro Scientific). With 1 μg of total RNA the synthesis of cDNA was performed using Superscript™II (Gibco-BRL) in a total volume of 20 μl. For RT-PCR on SCIM-1 and on housekeeping gene GAPDH, as positive control, 0.5 μl cDNA per reaction was used. PCR was performed in a Perkin Elmer 9600: 1 cycle 5 min 94° C., 35 cycles 30 sec 94° C./30 sec 55° C./1 min 72° C., 1 cycle 5 min 72° C. with 50 ng/primer, 200 μM dNTPs, and 2.5 u Taq polymerase (Pharmacia, #27-0799) in 25 μl total volume. Oligonucleotides specific for GAPDH were SEQ ID NO: 5 (5′CCCTTCATTGACCTCAACTACATGG) and SEQ ID NO: 6 (5′GGTCCACCACCCTGTTGCTGTAGCC). PCR samples were analysed on agarose gel (FIG. 1). Lanes 1-5 show clear signals of SCIM-1 cDNA amplification product of the expected length for the healthy cartilage (macroscopically) and 4/4 of the arthritis patients, while GAPDH-specific amplification signals are in the same order of magnitude among the RA cDNA preparations (lanes 7-10). The RT-PCR data indicate that the SCIM-1 gene is expressed in diseased tissue, i.e. afflicted knee cartilage, of 4/4 RA patients tested. It is likely that the SCIM-1 gene indeed is expressed in diseased articular cartilage of at least a considerable percentage of RA patients. Consequently, it is to be expected that the SCIM-1 protein is synthesised in diseased cartilage of RA patients.

### Example 3

SCIM-1 Gene Expression in Joint-Related Cells

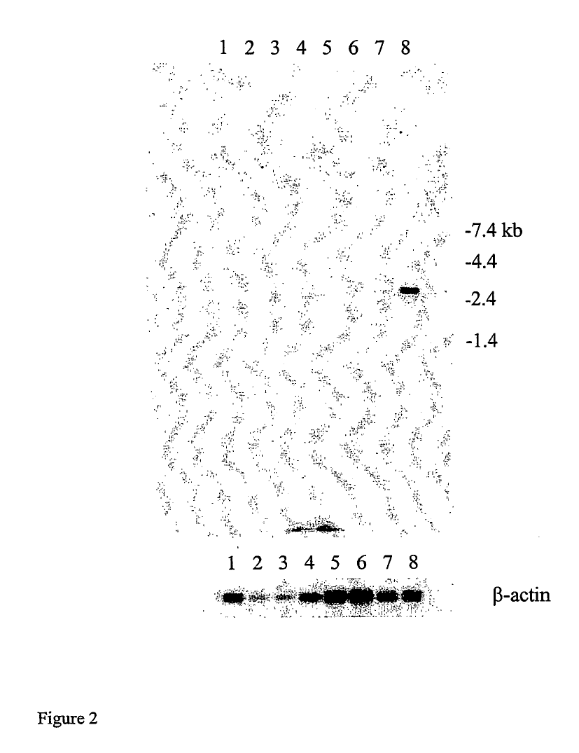

To determine tissue distribution of SCIM-1 gene expression multiple tissue Northern blots were probed with SCIM-1 cDNA fragments. Probes were generated via random primed labeling of SCIM-1 cDNA fragments nt(560-1048) and (1983-2427) (numbering according to SEQ ID NO: 1) using Ready-To-Go beads (Pharmacia) and is ³²P-α-dCTP (Amersham). Labeled probe was separated from free nucleotides on a 1 ml Sephadex G50-medium column and about 3×10⁶ cpm/ml of labeled probe was added to the hybridization mix (0.5 M phosphate buffer pH 7.0, 7% SDS, 1 mM EDTA) and hybridized to the Northern blot for 16 h at −65° C. Blots were washed up to 0.5×SSC at 65° C. and exposed to a STORM™840 Phosphor screen. Probe nt 560-1048 relative to SEQ ID NO: 1 hybridized weakly to a −2.4 kb mRNA in brain and a −3.0 kb mRNA in lung (Clontech human multiple tissue Northern blots H2 and H1, cat #7759-1 and 7760-1, respectively). Except for a clear signal at −2.6 kb in primary chondrocytes (FIG. 2, upper panel lanes 7-8) no signals were detected in other joint-related, cultured cells. Upon correction for the β-actin hybridization signal, as housekeeping gene control (FIG. 2, lower panel), the signal for SCIM-1 mRNA appeared to be enhanced with a factor 3-4 by TNFα (24 h, 10 ng/ml). With another SCIM-1 probe, i.e. nt(1983-2427), no signals were detected on Clontech's multiple tissue Northern blots H3 (cat #7767-1) and H2. Hybridization with probe nt(1983-2427) on RNA of primary chondrocytes yielded a signal for the same 2.6 kb mRNA as with probe nt(560-1048) (not shown), including the 3-4 fold upregulation of SCIM-1 gene expression by TNFα. Hybridization of a cDNA microarray with probes derived of chondrocytes incubated with or without IL-1, showed a 3-fold IL-1-dependent upregulation of SCIM-1 expression. Also for probe nt(1983-2427) no signals were observed on Northern blot for SV40-transformed human chondrocytes, which are likely to represent dedifferentiated cells. It was concluded that i) SCIM-1 gene expression displays a restricted tissue distribution, ii) the different lengths of detected mRNAs in brain, lung and chondrocytes could represent splice variants, and iii) SCIM-1 gene expression is enhanced by pro-inflammatory factors such as TNF and IL-1.

### Example 4

Isolation of the Full Length SCIM-1 cDNA

The complete coding sequence of human SCIM-1 was identified via SMART-RACE (#K1811-1, Clontech), using SCIM-1-specific oligonucleotides on RACE-cDNA that, was generated from 1 μg total RNA of TNFα-stimulated primary human chondrocytes. The chondrocytes were enzymatically (collagenase) isolated from healthy cartilage (knee), grown for about 3 weeks in monolayer culture (Hamm's/F12, 10% FCS) and subsequently stimulated with 10 ng/ml TNFα for 24 h. Oligonucleotides were designed based on sequences that were available for the two gene fragments identified from the Incyte database (Example 1). For the first gene fragment primers were: 5′RACE primer SEQ ID NO: 7 (5′GGGTCCATTGTACCCCGCCACGACG), and nested primer SEQ ID NO: 8 (5′CTCAAAGTCCCCATCATGGTCC), 3′RACE primer SEQ ID NO: 9 (5′CTCAGCCGCTGTCCGTCTTCCGG), and nested primer SEQ ID NO: 10 (5′GCTTCAACAACAACTGGCTGCG). For the second gene fragment primers were: 5′RACE primer SEQ ID NO: 11 (5′GGATGGGCTTGGGGAGGGTCTAGCTC), and nested primer SEQ ID NO: 12 (5′GCAGCAGCACAAGCCCACTTTC), 3′RACE primer SEQ ID NO: 13 (5′GTGCCCAGGGAGGTGGTGTCACTG), and nested primer SEQ ID NO: 14 (5′GCACAGGAAGTATGAGGACTTTAGTG). SMART-RACE PCRs were performed according to the Clontech manual PT3269-1 (March 1999). Sequencing of a number of RACE cDNA clones yielded the contig cDNA of SEQ ID NO: 1, which indicated that the two gene fragments as identified in Example 1, corresponded to the same gene, designated SCIM-1. A translation start codon was found at nt 59 and an open reading frame was found to encode 653 aa. With regard to the observed mRNA length of 2.6 kb in chondrocytes, the identified SCIM-1 cDNA of 2589 bp was considered very likely to contain the complete coding region of the corresponding gene.

Once the complete SCIM-1 cDNA had become available, a comparison was made with nucleic acid and protein sequences in Incyte and public domain databases, via blastsearches (NCBI-BLAST 2.0.10 Aug. 26, 1999; Altschul et al., Nucleic Acids Res. 25:3389-3402, 1997). Several public est sequences and a ˜1100 bp cDNA from patent application WO99/58660 were found to be identical to parts of the SCIM-1 cDNA. As a result of incomplete overlap, the complete SCIM-1 gene could not be reconstructed from these public cDNA fragments. Based on our complete encoded SCIM-1 amino acid sequence (SEQ ID NO: 2) a prediction was made as to functional protein domains of SCIM-1. A signal sequence was identified at aa 1-19 or 1-21, an integrin-binding RGD motif at aa 263-265, a calcium-binding EGF-like domain at aa 551-598, a putative hydrophobic region at aa 614-635, and MHC class II DR4Dw4 binding motifs at aa 12-20, 39-47, 149-157, and 323-331.

### Example 5

SCIM-1 Splice Variants and their Tissue Distribution

From multiple alignments (DNAman version 4.11) involving the full length SCIM-1 cDNA and all Incyte and public cDNAs that contained sequences identical to the SCIM-1 cDNA, it was discovered that the population of cDNAs was heterogeneous downstream of nucleotide 1852 (numbering according to SEQ ID NO: 1). This downstream region encodes for the C-terminal end of the protein, directly following the EGF-like domain. Four different C-terminal regions were identified for SCIM-1, i.e. variants A-D (Table 1; SEQ ID NO: 19-21, respectively, the C-terminal differences starting at amino acid position 599), deduced from cDNAs that were derived from different tissues (SEQ ID NO: 16-18, respectively; the 3′end differs as from nucleotide position 1853) and are expected to be the result of alternative splicing events. For the variants A, B, and C it is most likely that SEQ ID NO: 16-18 comprises the 3′ ends of the corresponding mRNAs, respectively, since each contains a 3′ poly(A) tail preceded by a putative poly-adenylation signal AATAAA. The different lengths of the 3′UTR regions for variants A and B are in agreement with the lengths of the SCIM-1 mRNAs that were detected on Northern blots for cartilage and brain tissues, respectively (see example 3). The number of cDNA clones that were found in the various databases to encode each of the variants A-D, strongly suggests that SCIM-1 variant A is predominantly expressed in joint tissues (cartilage/synovium) and to some extent in tumours and some other tissues, whereas expression of variant B seems to be restricted to tissues of the central nervous system (mostly brain). The suggested tissue-restricted expression was corroborated by an RT-PCR with oligonucleotides specific for the SCIM-1 variant B (SEQ ID NO: 9 and 15) on cDNA of total brain (Clontech human brain Quick-clone cDNA, cat. 7187-1/lot 9070843) and on cDNA of primary chondrocytes (cultured for 24 h with TNF-α, see example 4). Synthesis of cDNA and PCR conditions are as described in example 2. As a control PCR was performed with oligonucleotides specific for housekeeping gene GAPDH (SEQ ID NO: 5 and 6). A strong amplification signal of the expected length for the SCIM-1 variant B mRNA in brain tissue was seen whereas expression in chondrocytes hardly could be detected. The control PCR on GAPDH gene expression yielded a similar signal for both tissues, indicating that the input amounts of the corresponding cDNAs in the PCR were comparable. The finding that expression of variant B, represented by a 2.4 kb mRNA, was hardly detectable in the primary chondrocytes (24 h/TNF-α) is in agreement with the data of examples 3 and 4. On Northern blot only a single SCIM-1 mRNA variant of 2.6 kb was detected in primary chondrocytes (24 h/TNF-α) (FIG. 2, lane 8) which was concluded to represent variant A as 3′ RACE amplification on chondrocyte cDNA yielded only variant A sequences (see SEQ ID NO: 1).

### Example 6

Expression of Recombinant SCIM-1

The SCIM-1 (variant A) cDNA was cloned into cloning vector pCR2.1TOPO (Invitrogen) and subcloned as a Eco RI fragment to eukaryotic expression vector pNGV1 (EMBL accession number X99274). Consequently, the cDNA is situated behind the SV40 early promoter and a Kozak translation initition sequence. Upstream of the translational stopcodon 18 nucleotides were inserted encoding a His6-tag. In order to produce recombinant SCIM-1(His6) protein in eukaryotic cells, CHO cells (ATCC CCL61) were cultured in DMEM/Hamm's F12 containing 5% FCS (Harlan sera lab). The pNGV1-SCIM-1(His6) construct was transfected to CHO-K1 cells using Transfectam (Promega) and selection medium DMEAMamm's F12 containing 5% FCS and 0.8 mg/ml neomycin (G418 sulphate Gibco BRL Life technology, filter sterilised using a 0.22 μM Millipore SLGVO25BS filter). To detect recombinant protein, cells were cultured o/n in serum-free medium (containing 0.8 mg/ml neomycin) and expression was allowed to continue for one day. Serum-free culture supernatant was analysed on SDS-PAGE followed by Western blotting and subsequent detection with anti-His6 monoclonal antibody. The blot was blocked with 5% skimmed milk in P13S/0.05% Tween-20 and developed with a mouse-anti(His6)-tag antibody (Dianova GMBH cat. no. Dia 900. After three times washing with PBST for five minutes at RT, the blot was incubated with anti-mouse-IgG-HRP (Promega catno 3624512). After three times washing with PBST for five minutes at RT, detection was performed using a chromogenic substrate for HRP, i.e. diaminobezidine tetrahydrochloride in PBS, 2.5 MM CoCl₂ and 0.006% H₂O₂).

1. A polynucleotide encoding a polypeptide comprising the amino acid sequence extending from amino acid number 1-598 of SEQ ID NO: 2.

2. The polynucleotide according to claim 1 said polynucleotide encoding the amino acid sequence of SEQ ID NO: 2, SEQ ID NO: 19, SEQ ID NO: 20 or SEQ ID NO: 21.

3. The polynucleotide according to claim 1 or 2 said polynucleotide comprising the sequence extending from nucleotides 59-1852 of SEQ ID NO: 1.

4. The polynucleotide according to claims 1-3, said polynucleotide comprising the sequences SEQ ID NO: 1, SEQ ID NO: 16, SEQ ID NO: 17 or SEQ ID NO: 18 or the sequence extending from nucleotides 59-2017 from SEQ ID NO: 1, nucleotides 59-1969 from SEQ ID NO: 16, nucleotides 59-1912 from SEQ ID NO: 17 or nucleotides 59-1894 from SEQ ID NO: 18.

5. A recombinant expression vector comprising the DNA according to claims 1-4.

6. A polypeptide encoded by the polynucleotide according to claims 1-4, the expression vector according to claim 5 or immunomodulating fragments of SEQ ID NO: 2, SEQ ID NO: 19, SEQ ID NO: 20 or SEQ ID NO: 21.

7. Pharmaceutical composition comprising the polypeptide according to claim 6, and a pharmaceutical acceptable carrier.

8. The polypeptide according to claim 6 for use in therapy.

9. Use of the peptide according to claim 6 for the manufacture of a pharmaceutical preparation against inflammatory diseases.

10. Use of the polypeptide according to claims 6 for the manufacture of a pharmaceutical preparation for the induction of immunological tolerance to an autoantigen in patients suffering from autoimmune disorders, more specifically rheumatoid arthritis.

11. A cell transfected with DNA according to claims 1-4 or the expression vector according to claim 5.

12. A cell according to claim 11 which is a stable transfected cell which expresses the polypeptide according to claims 6.

13. Use of a DNA according to claims 1-4 or an expression vector according to claim 5, a cell according to claims 11 or 12 or a polypeptide according to claim 6 in a screening assay for identification of tolerogenic (poly)peptides.

14. A method for identifying tolerogenic peptides, said method comprising the steps of
a) introducing into a suitable host cell a fragment of the polynucleotide according to claims 1-4;
b) culturing the host cells under conditions to allow expression of the introduced sequence;
c) bringing the expression product in contact with lymphocytes; and
d) establishing the lymphocyte activity.

15. A method for identifying tolerogenic peptides, said method comprising the steps of
a) introducing into a suitable host cell a fragment of the polynucleotide according to claims 1-4;
b) culturing the host cells under conditions to allow expression of the introduced DNA sequence;
c) isolating the expression product;
d) bringing the expression product in contact with antigen presenting cells and T cells; and
e) establishing the T cell activity.

16. A method for identifying tolerogenic peptides, said method comprising the steps of
a) chemically synthesizing a fragment of SEQ ID NO: 2, SEQ ID NO: 19, SEQ ID NO: 20 or SEQ ID NO: 21;
b) bringing the peptide fragment in contact with antigen presenting cells and T cells; and
c) establishing the T cell activity.

17. A method for the formulation of a pharmaceutical composition comprising the method of claims 14-16 and mixing the peptide identified with a pharmaceutically acceptable carrier.

18. Use of a peptide as identified in the method of claims 14-16 for the preparation of a pharmaceutical suitable as a tolerogenic agent.

19. Diagnostic composition comprising one or more of the polypeptide according to claim 6 and a detection agent.

20. A diagnostic method for the detection of activated autoreactive T cells comprising the following steps:
a) isolation of the peripheral blood mononuclear cells (PBMC) from a blood sample of an individual,
b) culture of said PBMC under suitable conditions,
c) incubation of said PBMC culture in the presence of SCIM-1, fragments thereof and/or one or more peptides according to claim 6, and
d) detection of a response of T cells, indicating the presence of activated autoreactive T cells in the individual.

21. Test kit for the detection of activated autoreactive T cells, said test kit comprising SCIM-1 or one or more of the peptides according to claims 6.
